(19) United States
(12) Patent Application Publication
(10) Pub. No.: US 2008/0233167 A1
(43) Pub. Date: Sep. 25, 2008

(54) UROLOGICAL MEDICAL DEVICES FOR RELEASE OF PROSTATICALLY BENEFICIAL THERAPEUTIC AGENTS

(75) Inventors: Jianmin Li, Lexington, MA (US); Min-Shyan Sheu, Chelmsford, MA (US); Weenna Bucay-Couto, Burlington, MA (US)

Correspondence Address: MAYER & WILLIAMS PC, 251 NORTH AVENUE WEST, 2ND FLOOR, WESTFIELD, NJ, 07090, US

(73) Assignee: Boston Scientific Scimed, Inc., Maple Grove, MN (US)

(21) Appl. No.: 12/052,085

(22) Filed: Mar. 20, 2008

Related U.S. Application Data
(60) Provisional application No. 60/919,096, filed on Mar. 20, 2007.

(51) Int. Cl. A61F 2/04; A61M 25/00; A61M 1/00

(52) U.S. Cl. 424/423; 623/23.66; 604/523; 604/322

(57) ABSTRACT

According to an aspect of the invention, urological medical devices are provided, which comprise a prostatically beneficial agent selected from alpha-adrenergic blockers, antispasmodic agents, anticholinergic/antimuscarinic agents, calcium channel blockers, anti-inflammatory agents, hormone-affecting agents, anti-cancer agents, and combinations thereof, among others. The urological medical devices are adapted for implantation or insertion into a subject's urinary tract, whereupon at least a portion of the prostatically beneficial agent is released into the subject's prostatic urethra. The release profile of the prostatically beneficial agent is effective to treat a prostatic disorder, for example, benign prostate hypertrophy, prostate cancer or prostatitis, among others. Other aspects of the invention are directed to treating prostatic disorders.

### RELATED APPLICATION SECTION

This application claims the benefit of U.S. Provisional Application Ser. No. 60/919,096, filed Mar. 20, 2007, entitled “Urological Medical Devices for Release of Prostatically Beneficial Therapeutic Agents,” which is incorporated by reference herein in its entirety.

### FIELD OF THE INVENTION

The present invention relates generally to urological medical devices, and more particularly to implantable or insertable urological medical devices which release therapeutic agents.

### BACKGROUND OF THE INVENTION

With reference to FIG. 1, which depicts the male urogenital anatomy 10, the prostate 20 is a complex, walnut-sized gland in the male urogenital anatomy 10 that is located just below the bladder 22. The walls 23 of the bladder 22 relax and expand to store urine and contract and flatten to empty urine through the urethra 28, which extends from the bladder 23, through the prostate 20, and to the end of the penis 24. The part of the urethra 28 that is surrounded by the prostate 20 is referred to as the prostatic segment of the urethra, or prostatic urethra. The prostate 20 also surrounds the ejaculatory ducts where they enter the prostatic urethra 28. During sexual excitement, the sperm leave the epididymis 27 (which is attached to the surface of the testis 26) and is carried by the ductus deferens 29 in the direction of the prostate 20. A primary function of the prostate 20 is to supply nutritional fluid for the sperm to form semen during ejaculation.

The prostate is the site of various disorders. For example, a significant portion of the male populace sooner or later faces complaints related to the increased size of the prostate gland—a condition known as benign prostate hypertrophy (“BPH”). The predominant symptoms of BPH are an increase in frequency and urgency of urination, as well as retention of urine in the bladder, which eventually can lead to complete inability to urinate. The condition significantly alters the quality of life. Moreover, urinary retention inevitably leads to lower urinary tract infection, which ascends into the kidneys, leading to renal insufficiency and death, unless the cause (i.e., the BPH and its associated urine retention) is eliminated or at least abated.

Prostate cancer is the most common cancer amongst men in the United States and the second most common malignant cause of male death worldwide after lung cancer. The cause of prostate cancer is unknown, although some studies have shown a relationship between high dietary fat intake and increased testosterone levels. Regardless of the cause, prostate cancer is an increasingly significant global health problem in terms of mortality, morbidity, and economic impact.

Inflammatory disease of the prostate (prostatitis) is the most important disease of the prostate after BPH and cancer. This condition significantly interferes with the quality of life due to the presence of pain (prostodynia) and urethral discharge. Prostatitis can be treated with systemic antibiotic treatment, although the treatment period is lengthy and the recurrence rate high. This is partially due to the relative isolation of the prostate gland from the circulation, both anatomically as well as pharmacokinetically.

### SUMMARY OF THE INVENTION

According to an aspect of the present invention, urological medical devices are provided, which contain at least one prostatically beneficial agent selected from alpha-adrenergic blockers, antispasmodic agents, anticholinergic/antimuscarinic agents, calcium channel blockers, anti-inflammatory agents, hormone-affecting agents, anti-cancer agents, and combinations thereof, among others. The urological devices are adapted for implantation or insertion into a subject's urinary tract, whereupon at least a portion of the prostatically beneficial agent is released into the subject's prostatic urethra. The in vivo release profile of the prostatically beneficial agent (i.e., the quantity of drug that is released as a function of time) is effective to treat a prostatic disorder, for example, BPH, prostatitis or prostate cancer, among others.

According to another aspect of the present invention, a method of treating a prostatic disorder is provided which comprises: (a) identifying a subject with a prostatic disorder and (b) implanting or inserting into the subject a urological medical device which contains at least one prostatically beneficial agent. The medical device is adapted to release the at least one prostatically beneficial agent in the subject's prostatic urethra with a release profile that is effective to treat the prostatic disorder.

Further aspects include the following enumerated aspects, among others:

Aspect 1. A urological medical device comprising a prostatically beneficial agent selected from alpha-adrenergic blockers, antispasmodic agents, anticholinergic/antimuscarinic agents, calcium channel blockers, anti-inflammatory agents, hormone-affecting agents, anti-cancer agents, and combinations thereof, said urological medical device being adapted for implantation or insertion into a subject's urinary tract whereupon at least a portion of the prostatically beneficial agent is released within the subject's prostatic urethra with a release profile that is effective to treat a prostatic disorder selected from benign prostate hypertrophy, prostate cancer and prostatitis.

Aspect 2. The urological medical device of Aspect 1, wherein said urological medical device is an elongated solid device.

Aspect 3. The urological medical device of Aspect 1, wherein said urological medical device is an elongated hollow device.

Aspect 4. The urological medical device of Aspect 1, wherein said urological medical device is adapted to take on a coiled configuration within the subject.

Aspect 5. The urological medical device of Aspect 1, wherein said urological medical device is selected from a urethral stent, a catheter and a drainage tube.

Aspect 6. The urological medical device of Aspect 1, comprising a plurality of differing prostatically beneficial agents.

Aspect 7. The urological medical device of Aspect 1, wherein said prostatically beneficial agent is a calcium channel blocker.

Aspect 8. The urological medical device of Aspect 7, wherein said calcium channel blocker is selected from benzothiazepines, dihydropyridines, arylalkylamines, piperazines, and combinations thereof.

Aspect 9. The urological medical device of Aspect 7, wherein said calcium channel blocker is selected from diltiazem, nicardipine, nifedipine, nimodipine, bepridil, verapamil, mibefradil, pharmaceutically effective salts thereof, and combinations thereof.

Aspect 10. The urological medical device of Aspect 1, wherein said prostatically beneficial agent is an alpha-adrenergic blocker.

Aspect 11. The urological medical device of Aspect 1, wherein said prostatically beneficial agent is an alpha-1-adrenergic blocker.

Aspect 12. The urological medical device of Aspect 11, wherein said alpha-adrenergic blocker is selected from doxazosin, terazosin, pharmaceutically effective salts thereof, and combinations thereof.

Aspect 13. The urological medical device of Aspect 1, where said prostatically beneficial agent is an antispasmodic agent.

Aspect 14. The urological medical device of Aspect 1, where said antispasmodic agent is flavoxate or a pharmaceutically effective salt thereof.

Aspect 15. The urological medical device of Aspect 1, where said prostatically beneficial agent is an anticholinergic/antimuscarinic agent.

Aspect 16. The urological medical device of Aspect 1, where said anticholinergic/antimuscarinic agent is selected from oxybutynin, hyoscine, tolterodine, pharmaceutically effective salts thereof, and combinations thereof.

Aspect 17. The urological medical device of Aspect 1, where said prostatically beneficial agent is an anti-inflammatory agent.

Aspect 18. The urological medical device of Aspect 17, where said anti-inflammatory agent is a non-steroidal anti-inflammatory agent.

Aspect 19. The urological medical device of Aspect 1, where said prostatically beneficial agent is a hormone-affecting agent.

Aspect 20. The urological medical device of Aspect 19, wherein said hormone-affecting agent is selected from steroidal and nonsteroidal estrogens, steroidal and nonsteroidal antiandrogens, luteinising hormone releasing hormone analogs, gestrogens, and endothelin receptor antagonists, and combinations thereof.

Aspect 21. The urological medical device of Aspect 19, wherein said hormone-affecting agent is selected from diethylstilbestrol, estradiol, buserelin, goserelin, leuprolide, megestrol acetate, medroxyprogesterone acetate, ketoconazole and aminoglutethimide, bicalutamide, cyproterone, cuprotene acetate, flutamide, medroxyprogesterone acetate, nilutamide, atrasentan, pharmaceutically effective salts thereof, and combinations thereof.

Aspect 22. The urological medical device of Aspect 1, where said prostatically beneficial agent is an antineoplastic agent.

Aspect 23. The urological medical device of Aspect 22, where said antineoplastic agent is selected from antineoplastic antibiotics, alkaloids, nitrogen mustards, antimetabolites, and combinations thereof.

Aspect 24. The urological medical device of Aspect 22, where said antineoplastic agent is selected from doxorubicin, mitoxantrone, docetaxel, vinorelbine, gemcitabine, and combinations thereof.

Aspect 25. The urological medical device of Aspect 1, comprising a supplemental agent selected from corticosteroids, P-glycoprotein pump blockers, narcotic and non-narcotic analgesics, local anesthetic agents, antibiotics and combinations thereof, whereupon at least a portion of said supplemental agent is released in vivo.

Aspect 26. The urological medical device of Aspect 1, wherein said medical device comprises a polymeric carrier region that comprises said prostatically beneficial agent.

Aspect 27. The urological medical device of Aspect 26, wherein said polymeric carrier region corresponds to a urological medical device body.

Aspect 28. The urological medical device of Aspect 26, wherein said polymeric carrier region is in the form of a layer that at least partially covers an underlying urological medical device body.

Aspect 29. The urological medical device of Aspect 26, wherein said polymeric carrier region comprises a polymer selected from silicone polymers, polyurethanes, polyester polymers, and alkene polymers.

Aspect 30. The urological medical device of Aspect 26, wherein said polymeric carrier region comprises an alkene polymer selected from ethylene-vinyl acetate copolymers, ethylene-methacrylic acid copolymers, and ethylene-acrylic acid copolymers.

Aspect 31. The urological medical device of Aspect 26, wherein said polymeric carrier region comprises a biodegradable polymer.

Aspect 32. The urological medical device of Aspect 18, where said non-steroidal anti-inflammatory agent is selected from ketorolac and pharmaceutically acceptable salts thereof.

Aspect 33. A method of treating a prostatic disorder comprises: (a) identifying a subject with a prostatic disorder selected from benign prostate hypertrophy, prostate cancer and prostatitis and (b) implanting or inserting device of Aspect 1 into the subject.

Advantages of the present invention are that medical devices may be provided which, among other possible therapeutic benefits, treat various disorders of the prostate.

Another advantage of the present invention is that prostatically beneficial agents may be applied locally, thereby avoiding the need for systemic drug administration, which typically requires higher quantities of drug to be efficacious. In this regard, virtually all therapeutic agents have side effects.

These and other aspects, embodiments and advantages of the present invention will become immediately apparent to those of ordinary skill in the art upon review of the Detailed Description and Claims to follow.

### BRIEF DESCRIPTION OF THE DRAWING

FIG. 1 is a schematic representation of the male urogenital anatomy.

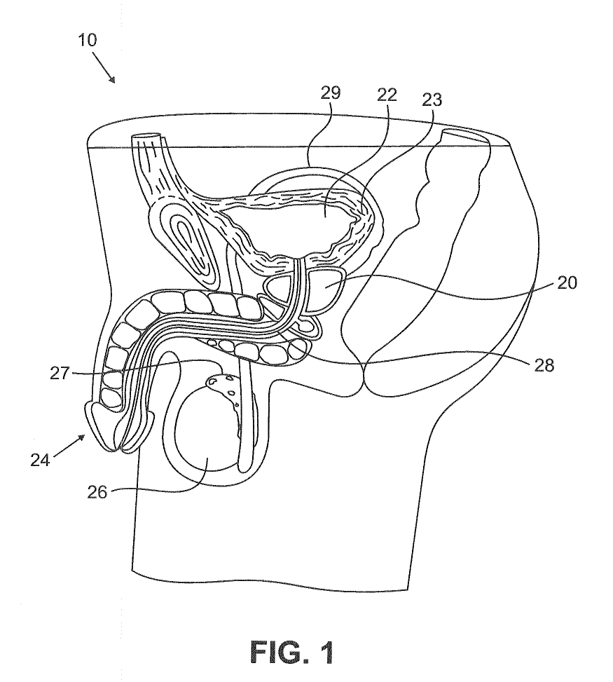

### DETAILED DESCRIPTION OF THE INVENTION

A more complete understanding of the present invention is available by reference to the following detailed description of numerous aspects and embodiments of the invention. The detailed description of the invention which follows is intended to illustrate but not limit the invention.

In one aspect, the present invention provides implantable or insertable urological medical devices, which are adapted to release at least one prostatically beneficial agent in a profile that is effective to treat prostatic disorders. As used herein, “treatment” (including variations thereof, for example, “treat,” “treating,” “treated,” etc.) refers to (i) the reduction or elimination of symptoms associated with a prostatic disorder (e.g., BPH, prostatitis, prostate cancer) and/or (ii) the substantial or complete elimination of a prostatic disorder. Preferred subjects (also referred to as “patients”) are vertebrate subjects, more preferably mammalian subjects and more preferably human subjects.

As used herein, a “prostatically beneficial agent” is an agent that is approved or capable of being approved by the United States Food and Drug Administration or Department of Agriculture as sufficiently safe and effective for use in treating prostatic disorders in humans or animals when released from an implantable or insertable urological medical device.

Urological medical devices for use in conjunction with the present invention include any device which can be positioned in the urinary tract of a subject and allows for the release of therapeutic agents within the prostatic urethra. These include various elongated devices including elongated devices having any of a variety of solid and hollow cross-sections including circular (e.g., tubular and rod-shaped devices), oval, triangular, and rectangular (e.g., ribbon-shaped devices), among many other regular and irregular cross sections. Specific examples include urological stents (e.g., urethral stents), urological catheters (e.g., drainage catheters, guide catheters, etc.), guidewires, urological scopes (e.g., cytoscopes, ureteroscopes, nephroscopes, etc.), patches, paving compositions, and injectable compositions, among others.

In some embodiments, devices are provided which are adapted to be advanced over a guide wire or advanced through a channel, for example, one associated with a guide catheter or scope.

In some embodiments, devices may be employed that take on a particular beneficial shape in vivo, for example, immediately upon removal of a guide wire or emergence from a channel (e.g., due to elastic rebound of the material) or upon application of an external stimulus such as heat or light (e.g., where a shape memory material such as a shape memory polymer is employed). For example, the device may take on a non-linear form such as a coiled configuration. Such constructions allow the medical device to be held in place in the urinary tract, for example, by forming a coil or other retention element in the bladder. Another example of a retention element is a balloon that is inflated in the bladder.

Prostatically beneficial agents for use in the invention may be selected, for example, from suitable members of the following: alpha-adrenergic blockers, antispasmodic agents, anticholinergic/antimuscarinic agents, calcium channel blockers, anti-inflammatory agents, hormone-affecting agents, anti-cancer agents, and combinations thereof, among others.

Examples of alpha-adrenergic blockers for use in the present invention may be selected from suitable members of the following: alfuzosin, amosulalol, arotinilol, dapiprazole, doxazosin, ergoloid mesylates, fenspiride, idazoxan, indoramin, labetalol, manotepil, naftopidil, nicergoline, prazosin, tamsulosin, terazosin, tolazoline, trimazosin, and yohimbine among others, as well as pharmaceutically acceptable salts, esters and other derivatives of the same, and combinations of the foregoing. Of these, tamsulosin, alfuzosin, doxazosin, terazosin, prazosin and tamsulosin are alpha-1-adrenergic blockers, of which tamsulosin and alfuzosin are selective alpha-1-adrenergic blockers.

Examples of antispasmodic agents for use in the present invention may be selected from suitable members of the following: alibendol, ambucetamide, aminopromazine, apoatropine, bevonium methyl sulfate, bietamiverine, butaverine, butropium, n-butylscopolammonium bromide, caroverine, cimetropium, cinnamedrine, clebopride, cyclonium iodide, difemerine, diisopromine, dioxaphetyl butyrate, diponium bromide, drofenine, emepronium bromide, ethaverine, etomidoline, feclemine, fenalamide, fenoverine, fenpiprane, fenpiverinium bromide, fentonium bromide, flavoxate, flopropione, gluconic acid, hydramitrazine, hymecromone, leiopyrrole, mebeverine, moxaverine, nafiverine, octamylamine, octaverine, pentapiperide, phenamacide, phloroglucinol, pinaverium, piperilate, pipoxolan hydrochloride, pramiverin, prifinium bromide, propyromazine, prozapine, racefemine, rociverine, sintropium bromide, spasmolytol, sultroponium, tiemonium iodide, tigloidine, tiquizium bromide, tiropramide, trepibutone, tricromyl, trifolium, trimebutine, n,n-ltrimethyl-3,3-diphen-ylpropylamine, tropenzile, trospium chloride, and xenotropium bromide, among others, as well as pharmaceutically acceptable salts, esters and other derivatives of the same, and combinations of the foregoing

Examples of anticholinergic/antimuscarinic agents for use in the present invention may be selected from suitable members of the following: adiphenine, alverine, ambutonomium, aminopentamide, amixetrine, amprotropine phosphate, anisotropine methylbromide, apoatropine, atropine, atropine n-oxide, benactyzine, benapryzine, benzetimide, benzilonium, benztropine mesylate, bevonium methyl sulfate, biperiden, butropium, n-butylscopolammonium bromide, buzepide, camylofine, caramiphen, chlorbenzoxamine, chlorphenoxamine, cimetropium, clidinium, cyclodrine, cyclonium, cycrimine, deptropine, dexetimide, dibutoline sulfate, dicyclomine, diethazine, difemerine, dihexyverine, diphemanil methylsulfate, n-(1,2-diphenylethyl)nicotinamide-, dipiproverine, diponium, emepronium, endobenzyline, ethopropazine, ethybenztropine, ethylbenzhydramine, etomidoline, eucatropine, fenpiverinium, fentonium, flutropium, glycopyrrolate, heteronium, hexocyclium methyl sulfate, homatropine, hyoscyamine, ipratropium, isopropamide, levomepate, mecloxamine, mepenzolate, metcaraphen, methantheline, methixene, methscopolamine, octamylamine, oxybutynin, oxyphencyclimine, oxyphenonium, pentapiperide, penthienate, phencarbamide, phenglutarimide, pipenzolate, piperidolate, piperilate, poldine methysulfate, pridinol, prifinium, procyclidine, propantheline, propenzolate, propiverine, propyromazine, scopolamine (hyoscine), scopolamine n-oxide, stramonium, sultroponium, thiphenamil, tiemonium, timepidium, tiquizium, tolterodine, tridihexethyl iodide, trihexyphenidyl hydrochloride, trimebutine, tropacine, tropenzile, tropicamide, trospium, valethamate, vamicamide, xenylropium, among others, as well as pharmaceutically acceptable salts, esters and other derivatives of the same, and combinations of the foregoing.

Examples of calcium channel blockers for use in the present invention may be selected from suitable members of the following, among others: arylalkylamines (including phenylalkylamines) such as verapamil, gallopamil, bepridil, clentiazen, fendiline, mibefradil, prenylamine, semotiadil, and terodiline; benzothiazepines such as diltiazem; dihydropyridine derivatives (including 1,4-dihydropyridine derivatives) such as amlodipine, aranidipine, barnidipine, benidipine, cilnidipine, efonidipine, elgodipine, felodipine, isradipine, lacidipine, lercanidipine, manidipine, nicardipine, nifedipine, nilvadipine, nimodipine, nisoldipine and nitrendipine; piperazine derivatives such as cinnarizine, dotarizine, flunarizine, lidoflazine and lomerizine; other calcium channel blockers such as bencyclane, etafenone, fantofarone, monatepil and perhexyline; as well as pharmaceutically acceptable salts, esters and other derivatives of the same, and combinations of the foregoing.

Anti-inflammatory agents include steroidal and non-steroidal anti-inflammatory agents. Examples of non-steroidal anti-inflammatory drugs for use in the present invention may be selected from suitable members of the following, among others: aminoarylcarboxylic acid derivatives such as enfenamic acid, etofenamate, flufenamic acid, isonixin, meclofenamic acid, mefanamic acid, niflumic acid, talniflumate, terofenamate and tolfenamic acid; arylacetic acid derivatives such as acemetacin, alclofenac, amfenac, bufexamac, cinmetacin, clopirac, diclofenac sodium, etodolac, felbinac, fenclofenac, fenclorac, fenclozic acid, fentiazac, glucametacin, ibufenac, indomethacin, isofezolac, isoxepac, lonazolac, metiazinic acid, oxametacine, proglumetacin, sulindac, tiaramide, tolmetin and zomepirac; arylbutyric acid derivatives such as bumadizon, butibufen, fenbufen and xenbucin; arylcarboxylic acids such as clidanac, ketorolac and tinoridine; arylpropionic acid derivatives such as alminoprofen, benoxaprofen, bucloxic acid, carprofen, fenoprofen, flunoxaprofen, flurbiprofen, ibuprofen, ibuproxam, indoprofen, ketoprofen, loxoprofen, miroprofen, naproxen, oxaprozin, piketoprofen, pirprofen, pranoprofen, protizinic acid, suprofen and tiaprofenic acid; pyrazoles such as difenamizole and epirizole; pyrazolones such as apazone, benzpiperylon, feprazone, mofebutazone, morazone, oxyphenbutazone, phenybutazone, pipebuzone, propyphenazone, ramifenazone, suxibuzone and thiazolinobutazone; salicylic acid and its derivatives such as acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamine o-acetic acid, salicylsulfuric acid, salsalate and sulfasalazine; thiazinecarboxamides such as droxicam, isoxicam, piroxicam and tenoxicam; others such as ε-acetamidocaproic acid, s-adenosylmethionine, 3-amino-4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole and tenidap; as well as pharmaceutically acceptable salts, esters and other derivatives of the same, and combinations of the foregoing.

Examples of steroidal anti-inflammatory agents (glucocorticoids) for use in the present invention may be selected from suitable members of the following: 21-acetoxyprefnenolone, aalclometasone, algestone, amicinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumehtasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol priopionate, halometasone, halopredone acetate, hydrocortamate, hydrocortisone, loteprednol etabonate, mazipredone, medrysone, meprednisone, methyolprednisolone, mometasone furoate, paramethasone, prednicarbate, prednisolone, prednisolone 25-diethylaminoacetate, prednisone sodium phosphate, prednisone, prednival, prednylidene, rimexolone, tixocortal, triamcinolone, triamcinolone acetonide, triamcinolone benetonide, triamcinolone hexacetonide, among others, as well as pharmaceutically acceptable salts, esters and other derivatives of the same, and combinations of the foregoing.

Examples of hormone-affecting agents for use in the present invention may be selected from suitable members of the following, among others: (a) nonsteroidal estrogens such as benzestrol, broparoestrol, chlorotrianisene, dienestrol, diethylstilbestrol, dimestrol, fosfestrol, hexestrol, methallenestril, and methestrol, (b) steroidal estrogens such as colpormon, conjugated estrogenic hormones, equilenin, equilin, estradiol, estriol, estrone, ethinyl estradiol, mestranol, moxestrol, mytatrienediol, quinestradiol, and quinestrol, (c) luteinising hormone releasing hormone (LHRH) analogs such as buserelin, deslorelin, goserelin, histrelin, leuprolide, nafarelin, and triptorelin, (d) other hormone-affecting agents, for example, gestrogens such as megestrol acetate and medroxyprogesterone acetate, ketoconazole and aminoglutethimide, (e) steroidal and nonsteroidal antiandrogens such as bicalutamide, bifluranol, cioteronel, cyproterone, cuprotene acetate, delmadinone acetate, flutamide, medroxyprogesterone acetate, nilutamide, osaterone, and oxendolone, and (f) endothelin receptor antagonists including atrasentan, zd4054, and ym598, as well as pharmaceutically acceptable salts, esters and other derivatives of the same, and combinations of the foregoing.

Examples of antineoplastic agents for use in the present invention may be selected from suitable members of the following, among others: (a) antineoplastic antibiotics such as aclacinomycins, actinomycin F1, anthramycin, azaserine, bleomycins, cactinomycin, carubicin, carzinophilin, chromomycins, dactinomycin, daunorubicin, 6-diazo-5-oxo-l-norleucine, doxorubicin, epirubicin, idarubicin, menogaril, mitoxantrone, mitomycins, mycophenolic acid, nogalamycin, olivomycins, peplomycin, pirarubicin, plicamycin, porfiromycin, puromycin, streptonigrin, streptozocin, tubercidin, zinostatin, and zorubicin, (b) alkaloids such as docetaxel, etoposide, irinotecan, paclitaxel, teniposide, topotecan, vinblastine, vincristine, vindesine and vinorelbine, (c) alkylating agents including nitrogen mustards, (d) antimetabolites including pyrimidine analogs such as gemcitabine, as well as pharmaceutically acceptable salts, esters and other derivatives of the same, and combinations of the foregoing.

Other prostatically beneficial agents have a combination of two or more of the above properties. One specific example of such an agent is estramustine phosphate sodium—a molecule in which estradiol and a nitrogen mustard are linked by a carbamate link.

In addition to one or more prostatically beneficial agents, the urological medical devices of the invention may also contain one or more optional supplemental agents (some of which may also have prostatically beneficial properties).

Such optional supplemental agents may, include, for example, supplemental therapeutic agents such as corticosteroids, P-glycoprotein pump blockers, narcotic and non-narcotic analgesics, local anesthetic agents, antibiotics and combinations thereof, among others. Such supplemental therapeutic agents may also be administered independently of urological devices of the invention, for example, by systemic administration or other local modes of administration.

Examples of corticosteroids for use in the present invention may be selected from suitable members of the following: betamethasone, cortisone, dexamethasone, deflazacort, hydrocortisone, methylprednisolone, prednisolone, prednisone and triamcinolone, among others, as well as pharmaceutically acceptable salts, esters and other derivatives of the same, and combinations of the foregoing.

Examples of P-glycoprotein pump blockers for use in the present invention may be selected from suitable members of the following: tariquidar (XR9576), zosuquidar, laniquidar and ONT-093, among others, as well as pharmaceutically acceptable salts, esters and other derivatives of the same, and combinations of the foregoing.

Examples of narcotic analgesic agents for use in the present invention may be selected from suitable members of the following: codeine, morphine, fentanyl, meperidine, propoxyphene, levorphanol, oxycodone, oxymorphone, hydromorphone, pentazocine, and methadone, among others, as well as pharmaceutically acceptable salts, esters and other derivatives of the same, and combinations of the foregoing.

Examples of non-narcotic analgesic agents for use in the present invention may be selected from suitable members of the following: analgesic agents such as acetaminophen, and non-steroidal anti-inflammatory drugs such as aspirin, diflunisal, salsalate, ibuprofen, ketoprofen, naproxen indomethacin, celecoxib, valdecoxib, diclofenac, etodolac, fenoprofen, flurbiprofen, ketorolac, meclofenamate, meloxicam, nabumetone, naproxen, oxaprozin, piroxicam, sulindac, tolmetin, and valdecoxib, among others, as well as pharmaceutically acceptable salts, esters and other derivatives of the same, and combinations of the foregoing.

Examples of local anesthetic agents for use in the present invention may be selected from suitable members of the following: benzocaine, cocaine, lidocaine, mepivacaine, and novacaine, among others, as well as pharmaceutically acceptable salts, esters and other derivatives of the same, and combinations of the foregoing.

Examples of antibacterial agents for use in the present invention may be selected from suitable members of the following: the penicillins (e.g., penicillin G, methicillin, oxacillin, ampicillin, amoxicillin, ticarcillin, etc.), the cephalosporins (e.g., cephalothin, cefazolin, cefoxitin, cefotaxime, cefaclor, cefoperazone, cefixime, ceftriaxone, cefuroxime, etc.), the carbapenems (e.g., imipenem, metropenem, etc.), the monobactems (e.g., aztreonem, etc.), the carbacephems (e.g., loracarbef, etc.), the glycopeptides (e.g., vancomycin, teichoplanin, etc.), bacitracin, polymyxins, colistins, fluoroquinolones (e.g., norfloxacin, lomefloxacin, fleroxacin, ciprofloxacin, enoxacin, trovafloxacin, gatifloxacin, etc.), sulfonamides (e.g., sulfamethoxazole, sulfanilamide, etc.), diaminopyrimidines (e.g., trimethoprim, etc.), rifampin, aminoglycosides (e.g., streptomycin, neomycin, netilmicin, tobramycin, gentamicin, amikacin, etc.), tetracyclines (e.g., tetracycline, doxycycline, demeclocycline, minocycline, etc.), spectinomycin, macrolides (e.g., erythromycin, azithromycin, clarithromycin, dirithromycin, troleandomycin, etc.), and oxazolidinones (e.g., linezolid, etc.), among others, as well as pharmaceutically acceptable salts, esters and other derivatives of the same, and combinations of the foregoing.

Many of the above and other prostatically beneficial agents and supplemental therapeutic agents may be found, for example, in The Merck Index, 13^{th} Edition, M. J. O'Neil, Senior Editor, published by Merck Research Laboratories, 2001.

Other examples of supplemental agents include imaging agents.

For example, x-ray based fluoroscopy is a diagnostic imaging technique that allows real-time patient monitoring of motion within a patient. To be fluoroscopically visible, devices and/or compositions are typically rendered more absorptive of x-rays than the surrounding tissue (e.g., radiopaque materials). In various embodiments of the invention, this is accomplished by the use of contrast agents. Examples of contrast agents for use in connection with x-ray fluoroscopy include metals, metal salts and oxides (particularly bismuth salts and oxides), and iodinated compounds, among others. More specific examples of such contrast agents include tungsten, platinum, tantalum, iridium, gold, or other dense metal, barium sulfate, bismuth subcarbonate, bismuth trioxide, bismuth oxychloride, metrizamide, iopamidol, iothalamate sodium, iodomide sodium, and meglumine, among others.

Ultrasound uses high frequency sound waves to create an image of living tissue. A sound signal is sent out, and the reflected ultrasonic energy, or “echoes,” are used to create the image. Ultrasound imaging contrast agents are materials that enhance the image produced by ultrasound equipment. Ultrasonic imaging contrast agents can be, for example, echogenic (i.e., materials that result in an increase in the reflected ultrasonic energy) or echolucent (i.e., materials that result in a decrease in the reflected ultrasonic energy). Suitable ultrasonic imaging contrast agents for use in connection with the present invention include solid particles ranging from about 0.01 to 50 microns in largest dimension (e.g., the diameter, where spherical particles are utilized), more typically about 0.5 to 20 microns. Both inorganic and organic particles can be used. Examples include microparticles/microspheres of calcium carbonate, hydroxyapatite, silica, poly(lactic acid), and poly(glycolic acid), among others. Microbubbles can also be used as ultrasonic imaging contrast agents, as is known in the imaging art.

Magnetic resonance imaging (MRI) produces images by differentiating detectable magnetic species in the portion of the body being imaged. In the case of ¹H MRI, the detectable species are protons (hydrogen nuclei). In order to enhance the differentiation of detectable species in the area of interest from those in the surrounding environment, imaging contrast agents are often employed. These agents alter the magnetic environment of the detectable protons in the area of interest relative to that of protons in the surrounding environment and thereby allow for enhanced contrast and better images of the area of interest. For contrast-enhanced MRI, it is desirable that the contrast agent have a large magnetic moment, with a relatively long electronic relaxation time. Based upon these criteria, contrast agents such as Gd(III), Mn(II) and Fe(III) have been employed. Gadolinium(III) has the largest magnetic moment among these three and is, therefore, a widely-used paramagnetic species to enhance contrast in MRI. Chelates of paramagnetic ions such as Gd-DTPA (gadolinium ion chelated with the ligand diethylenetriaminepentaacetic acid) have been employed as MRI contrast agents. Chelation of the gadolinium or other paramagnetic ion is believed to reduce the toxicity of the paramagnetic metal by rendering it more biocompatible, and can assist in localizing the distribution of the contrast agent to the area of interest. Further information can be found, for example, in U.S. Patent Application No. 2003/0100830 entitled “Implantable or insertable medical devices visible under magnetic resonance imaging,” the disclosure of which is incorporated herein by reference.

In certain embodiments of the invention, one or more agents (e.g., prostatically beneficial agents, optional supplemental agents such as supplemental therapeutic agents, supplemental imaging agents, etc.) are disposed within a polymeric carrier region. As used herein a polymeric carrier region is one that contains one or more polymers and one or more agents, which agent may or may not be released from the polymeric carrier region in vivo. The polymeric carrier region may correspond, for example, to an entire urological medical device or to a portion of a urological medical device. For instance, the polymeric carrier region may be in the form of a medical device body (e.g., a urethral stent body), in the form of a urological medical device component, in the form of one or more fibers which are incorporated into a urological medical device, or in the form of one or more polymeric layers formed over all or only a portion of an underlying substrate (e.g., a urological medical device body), among many other possibilities. Layers can be provided over an underlying substrate at a variety of locations and in a variety of shapes (e.g., in the form of a series of rectangles, stripes, or any other continuous or non-continuous pattern). As used herein a “layer” of a given material is a region of that material whose thickness is small compared to both its length and width. As used herein a layer need not be planar, for example, taking on the contours of an underlying substrate. Layers can be discontinuous (e.g., patterned). Terms such as “film,” “layer” and “coating” may be used interchangeably herein.

By “polymeric region” is meant a region (e.g., corresponding to a coating layer, a device component, a medical device body, etc.) that contains one or more types of polymers. By “carrier region” is meant a region that contains one or more agents, for example, selected from prostatically beneficial agents and optional supplemental agents such as those described above, among others. By “polymeric carrier region” is meant a region that contains one or more polymers and one or more agents.

As noted above, a “polymeric” region is one that contains polymers, for example, containing 50 wt % or less to 75 wt % to 90 wt % to 95 wt % to 97.5 wt % to 99 wt % or more polymers.

As used herein, “polymers” are molecules containing multiple copies (e.g., from 2 to 5 to 10 to 25 to 50 to 100 to 250 to 500 to 1000 or more copies) of one or more constitutional units, commonly referred to as monomers.

Polymers may take on a number of configurations, which may be selected, for example, from cyclic, linear, branched and networked (e.g., crosslinked) configurations. Branched configurations include star-shaped configurations (e.g., configurations in which three or more chains emanate from a single branch point, for instance an initiator molecule residue or a linking molecule residue), comb configurations (e.g., configurations having a main chain and a plurality of side chains), dendritic configurations (e.g., arborescent and hyperbranched polymers), and so forth.

As used herein, “homopolymers” are polymers that contain multiple copies of a single constitutional unit. “Copolymers” are polymers that contain multiple copies of at least two dissimilar constitutional units, examples of which include random, statistical, gradient, periodic (e.g., alternating) and block copolymers. As used herein, “block copolymers” are copolymers that contain two or more polymer blocks that differ in composition, for instance, because a constitutional unit (i.e., monomer) is found in one polymer block that is not found in another polymer block. As used herein, a “polymer block” is a grouping of constitutional units (e.g., 2 to 5 to 10 to 25 to 50 to 100 to 250 to 500 to 1000 or more units). Blocks can be branched or unbranched, and they may be networked (e.g., by crosslinking). Blocks can contain a single type of constitutional unit (also referred to herein as “homopolymeric blocks”) or multiple types of constitutional units (also referred to herein as “copolymeric blocks”) which may be provided, for example, in a random, statistical, gradient, or periodic (e.g., alternating) distribution.

Polymers for use in the present invention may be selected, for example, from various thermoplastic, elastomeric, and thermoplastic-elastomeric polymers.

Polymers for use in the present invention may be selected, for example, from silicone polymers, polyurethanes, silicone-polyurethane copolymers, polyesters, and alkene polymers.

Silicone polymers (also referred to as polysiloxanes) are polymers comprising one or more types of siloxane units,

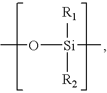

where R₁ and R₂ can be the same or different and may be selected from linear, branched and cyclic alkyl groups, aromatic groups and alky-aromatic groups, for example, having from 1 to 10 carbon atoms and having 2 or more, typically 5 to 10 to 25 to 50 to 100 to 250 to 500 to 1000 or more siloxane units. Examples include polydimethylsiloxane, polydiethylsiloxane, polymethylethylsiloxane, polymethylphenylsiloxane, and polydiphenylsiloxane, among many others.

In general, polyurethanes are a family of polymers that are synthesized from polyfunctional isocyanates (e.g., diisocyanates, including both aliphatic and aromatic diisocyanates) and polyols (also, referred to as macroglycols, e.g., macrodiols). Commonly employed macroglycols include polyester glycols, polyether glycols and polycarbonate glycols. Typically, aliphatic or aromatic diols are also employed as chain extenders, for example, to impart the useful physical properties described above. Examples of diol chain extenders include butane diol, pentane diol, hexane diol, heptane diol, benzene dimethanol, hydraquinone diethanol and ethylene glycol. Polyurethanes are commonly classified based on the type of macroglycol employed, with those containing polyester glycols being referred to as polyester polyurethanes, those containing polyether glycols being referred to as polyether polyurethanes, and those containing polycarbonate glycols being referred to as polycarbonate polyurethanes. Polyurethanes are also commonly designated aromatic or aliphatic on the basis of the chemical nature of the diisocyanate component in their formulation. For example, U.S. Patent App. No. 2004/0131863 to Belliveau et al. describes aliphatic polycarbonate polyurethanes which are the reaction products of (a) a hydroxyl terminated polycarbonate, (b) an aliphatic diisocyanate and (c) a lower aliphatic chain extender. Hydroxyl terminated polycarbonate polyol may be prepared by reacting a glycol with a carbonate, as disclosed in U.S. Pat. No. 4,131,731. Suitable aliphatic diisocyanates include hexamethylene diisocyanate (HDI), isophorone diisocyanate (IPDI), trimethyl hexamethylene diisocyanate (TMHDI), dicyclohexyl methane diisocyanate (HMDI), and dimer acid diisocyanate (DDI), with HMDI said to be preferred. Suitable chain extenders include lower aliphatic glycols having from about 2 to about 10 carbon atoms, such as, for instance ethylene glycol, diethylene glycol, propylene glycol, dipropylene glycol, 1,4-butanediol, 1,6-hexanediol, 1,3-butanediol, 1,5-pentanediol, 1,4-cyclohexanedimethanol hydroquinone di(hydroxyethyl)ether, neopentyglycol, and the like, with 1,4-butanediol said to be preferred.

Polyesters include, for example, polycarbonates and polyethylene terephthalate, among other polymers. Polycarbonates are derived from the reaction of carbonic acid derivatives with aromatic, aliphatic, or mixed diols. They may be produced, for example, by the reaction of phosgene with a diol in the presence of an appropriate hydrogen chloride receptor or by a melt transesterification reaction between a diol and a carbonate ester. Polycarbonates can be made from a wide variety of starting materials. For example, a common polycarbonate, bisphenol A polycarbonate, is a polycarbonate made by reacting bisphenol A with phosgene by condensation. For further information on polycarbnonates, see, e.g., U.S. Pat. No. 5,580,924 and the references cited therein. Polyethylene terephthalate may be produced for example, by the condensation reaction between ethylene glycol and terephalate or by the transesterification reaction between ethylene glycol and dimethyl terephthalate.

Silicone-polyurethane copolymers include Elast-Eon™ polymers (AorTech International plc).

Further polymers include alkene homopolymers and alkene copolymers, for example, copolymerized with themselves and/or with various other monomers including those selected from vinyl aromatic monomers such as styrene, acrylic acid, methacrylic acid, and vinyl acetate, among others. Examples of alkene monomers include ethylene, propylene, isobutylene, 1-butene, 1-pentene, 4-methyl-1-pentene, dienes such as 1,3-butadiene, 2-methyl-1,3-butadiene (isoprene), 2,3-dimethyl-1,3-butadiene, 2-ethyl-1,3-butadiene, 1,3-pentadiene, 2-methyl-1,3-pentadiene, 4-butyl-1,3-pentadiene, 2,3-dibutyl-1,3-pentadiene, 2-ethyl-1,3-pentadiene, 1,3-hexadiene, 1,3-octadiene, and 3-butyl-1,3-octadiene, among others. Specific examples of alkene copolymers include, poly(ethylene-co-vinyl acetate) (EVA), poly(ethylene-co-methacrylic acid), poly(ethylene-co-acrylic acid), and poly(isobutylene-co-styrene), among many others. Among EVA copolymers are included random and other copolymers having a vinyl acetate weight percent ratio of from about 0.5% to 1% to 2% to 5% to 15% to 20% to 30% to 40% or more. In general, the higher the vinyl acetate content, the lower the stiffness and Durometer of the EVA.

In certain embodiments, biodegradable polymers are employed in the present invention, which may include for example, polyesters, polyanhydrides, and/or amino acid based polymers, among others. Specific biodegradable polymers may be selected from suitable members of the following, among others: (a) polyester homopolymers and copolymers such as polyglycolide, poly-L-lactide, poly-D-lactide, poly-D,L-lactide, poly(beta-hydroxybutyrate), poly-D-gluconate, poly-L-gluconate, poly-D,L-gluconate, poly(epsilon-caprolactone), poly(delta-valerolactone), poly(p-dioxanone), poly(trimethylene carbonate), poly(lactide-co-glycolide) (PLGA), poly(lactide-co-delta-valerolactone), poly(lactide-co-epsilon-caprolactone), poly(lactide-co-beta-malic acid), poly(lactide-co-trimethylene carbonate), poly(glycolide-co-trimethylene carbonate), poly(beta-hydroxybutyrate-co-beta-hydroxyvalerate), poly[1,3-bis(p-carboxyphenoxy)propane-co-sebacic acid], poly(sebacic acid-co-fumaric acid), and poly(ortho esters) such as those synthesized by copolymerization of various diketene acetals and diols, among others, (b) polyanhydride homopolymers and copolymers such as poly(adipic anhydride), poly(suberic anhydride), poly(sebacic anhydride), poly(dodecanedioic anhydride), poly(maleic anhydride), poly[1,3-bis(p-carboxyphenoxy)methane anhydride], and poly[alpha,omega-bis(p-carboxyphenoxy)alkane anhydrides] such as poly[1,3-bis(p-carboxyphenoxy)propane anhydride] and poly[1,3-bis(p-carboxyphenoxy)hexane anhydride], among others; and (c) amino-acid-based homopolymers and copolymers including tyrosine-based polyarylates (e.g., copolymers of a diphenol and a diacid linked by ester bonds, with diphenols selected, for instance, from ethyl, butyl, hexyl, octyl and bezyl esters of desaminotyrosyl-tyrosine and diacids selected, for instance, from succinic, glutaric, adipic, suberic and sebacic acid), tyrosine-based polycarbonates (e.g., copolymers formed by the condensation polymerization of phosgene and a diphenol selected, for instance, from ethyl, butyl, hexyl, octyl and bezyl esters of desaminotyrosyl-tyrosine), and leucine and lysine-based polyester-amides; specific examples of tyrosine based polymers include poly(desaminotyrosyl-tyrosine ethyl ester adipate) or poly(DTE adipate), poly(desaminotyrosyl-tyrosine hexyl ester succinate) or poly(DTH succinate), poly(desaminotyrosyl-tyrosine ethyl ester carbonate) or poly(DTE carbonate), poly(desaminotyrosyl-tyrosine butyl ester carbonate) or poly(DTB carbonate), poly(desaminotyrosyl-tyrosine hexyl ester carbonate) or poly(DTH carbonate), and poly(desaminotyrosyl-tyrosine octyl ester carbonate) or poly(DTO carbonate).

In certain embodiments, hydrogel polymers are employed in the present invention. These include, for example, hydrogel polymers disclosed in U.S. Pat. Nos. 6,316,522, 6,261,630, 6,184,266, 6,176,849, 6,096,018, 6,060,534, 5,702,754, 5,693,034 and 5,304,121, the disclosures of which are hereby incorporated by reference. Specific examples of hydrogel polymers, not necessarily exclusive of the polymers in the prior paragraph, include polyacrylates, poly(acrylic acid), poly(metbacrylic acid), polyacrylamides, poly(N-alkylacrylamides), polyalkylene oxides such as poly(ethylene oxide) and poly(propylene oxide), poly(vinyl alcohol), poly(vinyl aromatics), poly(vinylpyrrolidone), poly(ethylene imine), poly(ethylene amine), polyacrylonitrile, poly(vinyl sulfonic acid), polyamides, poly(L-lysine), hydrophilic polyurethanes, maleic anhydride polymers, proteins, collagen, cellulosic polymers, methyl cellulose, carboxymethyl cellulose, dextran, carboxymethyl dextran, modified dextran, alginates, alginic acid, pectinic acid, hyaluronic acid, chitin, pullulan, gelatin, gellan, xanthan, carboxymethyl starch, chondroitin sulfate, guar, starch, and blends, copolymers, and derivatives thereof, among others.

Various methods of crosslinking hydrogel polymers are known and include, for instance, (a) covalent crosslinking, for example, with polyfunctional crosslinking agents that bridge hydrogel polymer chains by reaction with functional groups along the hydrogel polymer chains and/or (b) ionic crosslinking, for example, using polyvalent ions. Other crosslinking methods, such as crosslinking by exposing the hydrogel polymer to light of an appropriate frequency, may also be employed. Thus, hydrogel polymers useful in accordance with the present invention may be ionically crosslinked, covalently crosslinked, ionically and covalently crosslinked, or crosslinked by other methods known in the art. A polyfunctional crosslinking agent may be any compound having at least two functional groups that react with functional groups in the hydrogel polymer. Crosslinking ions that are used to ionically crosslink the hydrogel polymers may be anions or cations, depending on whether the polymer is anionically or cationically crosslinkable. Covalent and ionic crosslinking agents are well known in the hydrogel art.

Further polymers for use in the present invention may be selected, for example, from suitable members of the following (which polymers are not necessarily exclusive of those described above): polycarboxylic acid polymers and copolymers including polyacrylic acids; acetal polymers and copolymers; acrylate and methacrylate polymers and copolymers (e.g., n-butyl methacrylate); cellulosic polymers and copolymers, including cellulose acetates, cellulose nitrates, cellulose propionates, cellulose acetate butyrates, cellophanes, rayons, rayon triacetates, and cellulose ethers such as carboxymethyl celluloses and hydroxyalkyl celluloses; polyoxymethylene polymers and copolymers; polyimide polymers and copolymers such as polyether block imides and polyether block amides, polyamidimides, polyesterimides, and polyetherimides; polysulfone polymers and copolymers including polyarylsulfones and polyethersulfones; polyamide polymers and copolymers including nylon 6,6, nylon 12, polycaprolactams and polyacrylamides; resins including alkyd resins, phenolic resins, urea resins, melamine resins, epoxy resins, allyl resins and epoxide resins; polycarbonates; polyacrylonitriles; polyvinylpyrrolidones (cross-linked and otherwise); polymers and copolymers of vinyl monomers including polyvinyl alcohols, polyvinyl halides such as polyvinyl chlorides, ethylene-vinyl acetate copolymers (EVA), polyvinylidene chlorides, polyvinyl ethers such as polyvinyl methyl ethers, polystyrenes, styrene-maleic anhydride copolymers, vinyl-aromatic-alkylene copolymers, including styrene-butadiene copolymers, styrene-ethylene-butylene copolymers (e.g., a polystyrene-polyethylene/butylene-polystyrene (SEBS) copolymer, available as Kraton® G series polymers), styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers (e.g., polyisobutylene-polystyrene and polystyrene-polyisobutylene-polystyrene block copolymers such as those disclosed in U.S. Pat. No. 6,545,097 to Pinchuk), polyvinyl ketones, polyvinylcarbazoles, and polyvinyl esters such as polyvinyl acetates; polybenzimidazoles; ethylene-methacrylic acid copolymers and ethylene-acrylic acid copolymers, where some of the acid groups can be neutralized with either zinc or sodium ions (commonly known as ionomers); polyalkyl oxide polymers and copolymers including polyethylene oxides (PEO); polyesters including polyethylene terephthalates and aliphatic polyesters such as polymers and copolymers of lactide (which includes lactic acid as well as d-,l- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of poly(lactic acid) and poly(caprolactone) is one specific example); polyether polymers and copolymers including polyarylethers such as polyphenylene ethers, polyether ketones, polyether ether ketones; polyphenylene sulfides; polyisocyanates; polyolefin polymers and copolymers, including polyalkylenes such as polypropylenes, polyethylenes (low and high density, low and high molecular weight), polybutylenes (such as polybut-1-ene and polyisobutylene), polyolefin elastomers (e.g., santoprene), ethylene propylene diene monomer (EPDM) rubbers, poly-4-methyl-pen-1-enes, ethylene-alpha-olefin copolymers, ethylene-methyl methacrylate copolymers and ethylene-vinyl acetate copolymers; fluorinated polymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF); silicone polymers and copolymers; thermoplastic polyurethanes (TPU); elastomers such as elastomeric polyurethanes and polyurethane copolymers (including block and random copolymers that are polyether based, polyester based, polycarbonate based, aliphatic based, aromatic based and mixtures thereof; examples of commercially available polyurethane copolymers include Bionate®, Carbothane®, Tecoflex®, Tecothane®, Tecophilic®, Tecoplast®, Pellethane®, Chronothane® and Chronoflex®; p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; biopolymers, such as polypeptides, proteins, polysaccharides and fatty acids (and esters thereof), including fibrin, fibrinogen, collagen, elastin, chitosan, gelatin, starch, glycosaminoglycans such as hyaluronic acid; as well as blends, further copolymers and derivatives of the above, among others.

A wide range of agent loadings (e.g., selected from loadings of prostatically beneficial agents and optional supplemental agents such as optional therapeutic agents, imaging agents, etc.) may be used in conjunction with the urological medical devices of the present invention, with the effective amount being readily determined by those of ordinary skill in the art. For a polymeric carrier region, typical loadings range, for example, from than 1 wt % or less to 2 wt % to 5 wt % to 10 wt % to 25 wt % to 50 wt % or more.

The release profile of the one or more prostatically beneficial agents from the device (as well as the release profile of any optional supplemental agents), will be affected by a number of variables. For example, where a polymeric carrier region is utilized, the release profile will depend upon the particular agent(s) selected, the particular polymer(s) that are selected, and their relative amounts. The release profile will also be affected by the size, number and/or position of the polymeric carrier regions within the device. For example, the release profile may be modified by varying the thickness or surface area of the polymeric carrier region. Moreover, multiple polymeric carrier regions may be employed. For example, multiple polymeric carrier regions having the same or different content (e.g., different polymeric content and/or different agent content) may be positioned laterally with respect to one another. Alternatively, a polymeric layer (e.g., formed from one or more polymers described above, either with or without additional agents) may be positioned over a polymeric carrier region in accordance with the invention, thereby acting as a barrier layer.

In some embodiments, the release profile may be modified by increasing the rate at which the polymeric region absorbs water from the surrounding environment, for example, by employing a rapidly hydrating polymer (e.g., a hydrogel) or a rapidly hydrating polymer block (or by varying the ratio of a rapidly hydrating polymer or polymer block vis-à-vis a slowly hydrating polymer or polymer block, respectively), by the addition of an osmotic agent such as a soluble salt or sugar excipient as an optional supplemental agent, and so forth.

Numerous techniques are available for forming polymeric carrier regions in accordance with the present invention.

For example, where the polymeric carrier region is formed from one or more polymers having thermoplastic characteristics, a variety of standard thermoplastic processing techniques may be used to form the polymeric carrier region, including injection molding, compression molding, blow molding, spinning, vacuum forming and calendaring, extrusion into sheets, fibers, rods, tubes and other cross-sectional profiles of various lengths, and combinations of these processes. Using these and other thermoplastic processing techniques, entire devices or portions thereof can be formed.

In some embodiments, one or more polymers making up the carrier region and one or more agents (e.g., selected from prostatically beneficial agents and optional supplemental agents) may be mixed or compounded using any suitable processing technique known in the art. For example, where thermoplastic materials are employed, a polymer melt may be formed. A common way of doing so is to apply mechanical shear to a mixture of the polymer(s) and the agent(s). After compounding, the material may be processed using, for example, one or more of the thermoplastic techniques described above, among others.

Other processing techniques besides thermoplastic processing techniques may also be used to form the polymeric carrier regions of the present invention, including solvent-based techniques. Using these techniques, a polymeric carrier region can be formed by (a) first providing a solution or dispersion that contains (i) solvent, (ii) polymer(s), (iii) prostatically beneficial agent(s), and (iv) any optional supplemental agent(s) and (b) subsequently removing the solvent. The solvent that is ultimately selected will contain one or more solvent species (e.g., water and/or one or more organic solvents), which are generally selected based on their ability to dissolve the polymer(s) that form the polymeric carrier region (and in many embodiments the prostatically beneficial agent(s) and any optional supplemental agent(s)), in addition to other factors, including drying rate, surface tension, etc. Preferred solvent-based techniques include, but are not limited to, solvent casting techniques, solvent spraying techniques, spin coating techniques, web coating techniques, dipping techniques, techniques involving coating via mechanical suspension including air suspension, ink jet techniques, electrostatic techniques, and combinations of these processes.

In certain embodiments of the invention, a polymer-containing solution (where solvent-based processing is employed) or a polymer melt (where thermoplastic processing is employed) is applied to a substrate to form a polymeric carrier region, which solution or melt may also contain prostatically beneficial agent(s) and/or any optional supplemental agent(s). For example, the substrate can correspond to all or a portion of an implantable or insertable urological medical device body to which a polymeric carrier region is applied. The substrate can also be, for example, a template, such as a mold, from which the polymeric carrier region is removed after solidification. In certain other embodiments, for example, extrusion and co-extrusion techniques, one or more polymeric carrier regions are formed without the aid of a substrate. In a more specific example, an entire stent body may be extruded as a carrier region. In another, a polymeric carrier layer may be co-extruded along with an underlying stent body. In another, a polymeric carrier layer may be provided by spraying or extruding a coating layer onto a pre-existing stent body. In yet another more specific example, a stent body may be cast in a mold.

As seen from the above, where various agents—for example, prostatically beneficial agent(s) and/or any optional supplemental agent(s)—are stable under the polymer processing conditions employed, then they can be combined with the polymers and co-processed along with the same to form the polymeric carrier region of interest. Alternatively, the agents can be introduced subsequent to the formation of the polymeric region using techniques such as imbibing (e.g., where the agent or agents of choice are dissolved or dispersed in a solvent and then contacted with the device, for instance, by spraying, dipping, etc.).

In certain embodiments, the polymeric carrier regions may be crosslinked using methods known in the art, for example, to render them water insoluble.

As noted above, at least one polymeric barrier layer may be provided over a carrier region in accordance with an embodiment of the invention. Such barrier layers may be formed, for example, from the polymer listed above, among others. In these embodiments, the polymeric barrier layer may be formed over the carrier region, for example, using one of the solvent based or thermoplastic techniques described above. Alternatively, a previously formed polymeric barrier region may be adhered over a carrier region.

Although various embodiments are specifically illustrated and described herein, it will be appreciated that modifications and variations of the present invention are covered by the above teachings and are within the purview of the appended claims without departing from the spirit and intended scope of the invention.

1. A urological medical device comprising a prostatically beneficial agent selected from alpha-adrenergic blockers, antispasmodic agents, anticholinergic/antimuscarinic agents, calcium channel blockers, anti-inflammatory agents, hormone-affecting agents, anti-cancer agents, and combinations thereof, said urological medical device being adapted for implantation or insertion into a subject's urinary tract whereupon at least a portion of the prostatically beneficial agent is released within the subject's prostatic urethra with a release profile that is effective to treat a prostatic disorder selected from benign prostate hypertrophy, prostate cancer and prostatitis.

2. The urological medical device of claim 1, wherein said urological medical device is an elongated solid device.

3. The urological medical device of claim 1, wherein said urological medical device is an elongated hollow device.

4. The urological medical device of claim 1, wherein said urological medical device is adapted to take on a coiled configuration within the subject.

5. The urological medical device of claim 1, wherein said urological medical device is selected from a urethral stent, a catheter and a drainage tube.

6. The urological medical device of claim 1, comprising a plurality of differing prostatically beneficial agents.

7. The urological medical device of claim 1, wherein said prostatically beneficial agent is a calcium channel blocker.

8. The urological medical device of claim 7, wherein said calcium channel blocker is selected from benzothiazepines, dihydropyridines, arylalkylamines, piperazines, and combinations thereof.

9. The urological medical device of claim 7, wherein said calcium channel blocker is selected from diltiazem, nicardipine, nifedipine, nimodipine, bepridil, verapamil, mibefradil, pharmaceutically effective salts thereof, and combinations thereof.

10. The urological medical device of claim 1, wherein said prostatically beneficial agent is an alpha-adrenergic blocker.

11. The urological medical device of claim 1, wherein said prostatically beneficial agent is an alpha-1-adrenergic blocker.

12. The urological medical device of claim 11, wherein said alpha-adrenergic blocker is selected from doxazosin, terazosin, pharmaceutically effective salts thereof, and combinations thereof.

13. The urological medical device of claim 1, where said prostatically beneficial agent is an antispasmodic agent.

14. The urological medical device of claim 1, where said antispasmodic agent is flavoxate or a pharmaceutically effective salt thereof.

15. The urological medical device of claim 1, where said prostatically beneficial agent is an anticholinergic/antimuscarinic agent.

16. The urological medical device of claim 1, where said anticholinergic/antimuscarinic agent is selected from oxybutynin, hyoscine, tolterodine, pharmaceutically effective salts thereof, and combinations thereof.

17. The urological medical device of claim 1, where said prostatically beneficial agent is an anti-inflammatory agent.

18. The urological medical device of claim 17, where said anti-inflammatory agent is a non-steroidal anti-inflammatory agent.

19. The urological medical device of claim 1, where said prostatically beneficial agent is a hormone-affecting agent.

20. The urological medical device of claim 19, wherein said hormone-affecting agent selected from steroidal and nonsteroidal estrogens, steroidal and nonsteroidal antiandrogens, luteinising hormone releasing hormone analogs, gestrogens, and endothelin receptor antagonists, and combinations thereof.

21. The urological medical device of claim 19, wherein said hormone-affecting agent is selected from diethylstilbestrol, estradiol, buserelin, goserelin, leuprolide, megestrol acetate, medroxyprogesterone acetate, ketoconazole and aminoglutethimide, bicalutamide, ryproterone, cuprotene acetate, flutamide, medroxyprogesterone acetate, nilutamide, atrasentan, pharmaceutically effective salts thereof, and combinations thereof.

22. The urological medical device of claim 1, where said prostatically beneficial agent is an antineoplastic agent.

23. The urological medical device of claim 22, where said antineoplastic agent is selected from antineoplastic antibiotics, alkaloids, nitrogen mustards, antimetabolites, and combinations thereof.

24. The urological medical device of claim 22, where said antineoplastic agent is selected from doxorubicin, mitoxantrone, docetaxel, vinorelbine, gemcitabine, and combinations thereof.

25. The urological medical device of claim 1, comprising a supplemental agent selected from corticosteroids, P-glycoprotein pump blockers, narcotic and non-narcotic analgesics, local anesthetic agents, antibiotics and combinations thereof, whereupon at least a portion of said supplemental agent is released in vivo.

26. The urological medical device of claim 1, wherein said medical device comprises a polymeric carrier region that comprises said prostatically beneficial agent.

27. The urological medical device of claim 26, wherein said polymeric carrier region corresponds to a urological medical device body.

28. The urological medical device of claim 26, wherein said polymeric carrier region is in the form of a layer that at least partially covers an underlying urological medical device body.

29. The urological medical device of claim 26, wherein said polymeric carrier region comprises a polymer selected from silicone polymers, polyurethanes, polyester polymers, and alkene polymers.

30. The urological medical device of claim 26, wherein said polymeric carrier region comprises an alkene polymer selected from ethylene-vinyl acetate copolymers, ethylene-methacrylic acid copolymers, and ethylene-acrylic acid copolymers.

31. The urological medical device of claim 26, wherein said polymeric carrier region comprises a biodegradable polymer.

32. The urological medical device of claim 18, where said non-steroidal anti-inflammatory agent is selected from ketorolac and pharmaceutically acceptable salts thereof.

33. A method of treating a prostatic disorder comprises: (a) identifying a subject with a prostatic disorder selected from benign prostate hypertrophy, prostate cancer and prostatitis and (b) implanting or inserting device of claim 1 into the subject.
